SEQUENCE LISTING (1 sequence)

SEQ ID NO: 1 - protein, 299 aa, synthetic construct
  REGION 1..299: note = Synthetic Construct
FVNQHLCGSHLVEALELVCGERGFHYGGGGGGSGGGGGIVEQCCTSTCSLDQLENYCGGGGGQGGGGQGGGGQGGGGGECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

(19) United States
(12) Patent Application Publication
(10) Pub. No.: US 2025/0000950 A1
(43) Pub. Date: Jan. 2, 2025

(54) PRESERVED FORMULATIONS

(71) Applicant: ELI LILLY AND COMPANY, Indianapolis, IN (US)

(72) Inventors: David Paul ALLEN, Carmel, IN (US); John Michael BEALS, Solana Beach, CA (US); Vincent John CORVARI, Carmel, IN (US); Patrick Daniel DONOVAN, Zionsville, IN (US); Ken Kangyi QIAN, Carmel, IN (US); Wei WANG, Zionsville, IN (US)

(21) Appl. No.: 18/709,721

(22) PCT Filed: Nov. 14, 2022
(86) PCT No.: PCT/US2022/079791

§ 371 Date: May 13, 2024

Related U.S. Application Data
(60) Provisional application No. 63/279,390, filed on Nov. 15, 2021.

(51) Int. Cl. A61K 38/28; A61K 47/02; A61K 47/10

(52) U.S. Cl. CPC A61K 38/28; A61K 47/02; A61K 47/10

(57) ABSTRACT

Described herein are preserved formulations of insulin-Fc fusions. The formulations include insulin-Fc fusions having prolonged pharmacokinetic and pharmacodynamic profiles sufficient for once weekly administration in the treatment of diabetes and are sufficiently stable to allow for storage and use without unacceptable loss of chemical or physical stability.

The present invention relates to preserved formulations of insulin-Fc fusions. The formulations include insulin-Fc fusions having prolonged pharmacokinetic and pharmacodynamic profiles sufficient for once weekly administration in the treatment of diabetes and are sufficiently stable to allow for storage and use without unacceptable loss of chemical or physical stability.

Diabetes is a chronic disorder characterized by hyperglycemia resulting from defects in insulin secretion, insulin action, or both. Type 1 diabetes (TID) is characterized by little or no insulin secretory capacity, and patients with TID require insulin therapy for survival. Type 2 diabetes (T2D) is characterized by elevated blood glucose levels resulting from impaired insulin secretion, insulin resistance, excessive hepatic glucose output, and/or contributions from all of the above. In many patients with T2D, the disease progresses to a requirement for insulin therapy.

Because TID patients produce little or no insulin, effective insulin therapy generally involves the use of two types of exogenously administered insulin: a rapid-acting, mealtime insulin provided by bolus injections, and a long-acting, basal insulin, administered once or twice daily to control blood glucose levels between meals. Treatment of patients with T2D typically begins with prescribed weight loss, exercise, and a diabetic diet, but when these measures fail to control elevated blood sugars, then oral medications and incretin-based therapy may be necessary. When these medications are still insufficient, treatment with insulin is considered. T2D patients whose disease has progressed to the point that insulin therapy is required are generally started on a single daily injection of a long-acting, basal insulin.

Basal insulins currently available include insulin glargine, sold under the tradename LANTUSR, insulin detemir, sold under the tradename LEVEMIRR), and insulin degludec, sold under the tradename TRESIBA®. These insulins are each indicated for once-daily administration and are available in preserved formulations that have sufficient antimicrobial effectiveness to allow for multiple doses to be administered from a single container or device.

Treatment regimens involving daily injections of existing insulin therapies can be complicated and painful to administer and can result in undesired side effects, such as hypoglycemia and weight gain. Research is being conducted to develop insulin products with longer duration of action; thus, requiring fewer injections than currently available insulin products, including as infrequently as once-weekly.

One category of such insulin products comprises moieties that activate the insulin receptor attached to Fc regions of an antibody, referred to herein as insulin-Fc fusions. Examples of such products are described in U.S. Pat. No. 9,855,318, which describes compounds and formulations thereof, including formulations comprising the phenolic preservative m-cresol, which is commonly used in insulin products, including the once-daily basal insulins described above.

It has been found, however, that formulations of insulin-Fc fusions with the concentrations of preservatives described in U.S. Pat. No. 9,855,318 and/or in currently available insulin products may lead to unacceptable stability liabilities. Thus, there is a need for new formulations with preservatives that provide sufficient antimicrobial effectiveness but that do not result in unacceptable stability liabilities.

The present invention seeks to meet those needs.

Accordingly, in one aspect the present invention provides an aqueous, sterile pharmaceutical composition comprising:
a) an insulin-Fc fusion;
b) phenol;
c) one or more additional preservatives selected from the group consisting of phenoxyethanol and benzyl alcohol;
d) a tonicity agent;
e) a surfactant;
f) a buffer; and
having a pH between 6 to 7.5; and
   wherein the phenol and one or more additional preservatives are present in concentrations that allow for an in-use period of at least 12 weeks without unacceptable loss of stability.

In another aspect, the present invention provides an aqueous, sterile pharmaceutical composition comprising:
a) basal BIF in a concentration between 5-30 mg/mL;
b) phenol in a concentration of 1.5 to 4 mg/mL;
c) benzyl alcohol in a concentration between 4 to 14 mg/mL;
d) glycerin in a concentration of 15 to 35 mg/mL;
e) poloxamer 188 in a concentration of 0.01 to 0.5 mg/mL; and
f) phosphate in a concentration of 5-10 mM:
   wherein the composition has a pH of 6 to 7.5.

In another aspect, the present invention provides a method of improving glycemic control comprising administering to a human in need thereof an effective dose of an aqueous, sterile pharmaceutical composition of the present invention.

In addition, the present invention provides an aqueous, sterile pharmaceutical composition of the present invention for use in therapy. More particularly, the present invention provides a pharmaceutical composition for use in improving glycemic control. The present invention also provides the use of a pharmaceutical composition in the manufacture of a medicament for improving glycemic control.

In addition, the present invention provides an article of manufacture comprising an aqueous, sterile pharmaceutical composition of the present invention. More particularly, in certain aspects the article of manufacture is a multi-use vial, a cartridge, a re-usable pen injector, a disposable pen device, a pump device for continuous subcutaneous insulin infusion therapy or a container closure system for use in a pump device for continuous subcutaneous insulin infusion therapy.

The present invention is directed to preserved formulations of insulin-Fc fusions that have prolonged duration of action. Insulin-Fc fusions have been described for example in U.S. Pat. No. 9,855,318: CN103509118: WO2011/122921: US2015/0196643: WO2018/185131: WO2020/006529: WO2020/074544: WO2021126584: US20210300983: US2021/0324033; and US2021340212.

In certain preferred embodiments, the insulin-Fc fusion is a compound described in U.S. Pat. No. 9,855,318 known as basal insulin Fc (BIF) or insulin efsitora alfa (CAS registry number 2131038 Nov. 2). BIF comprises a dimer of an insulin receptor agonist fused to a human IgG Fc region, wherein the insulin receptor agonist comprises an insulin B-chain analog fused to an insulin A-chain analog through the use of a first peptide linker and wherein the C-terminal residue of the insulin A-chain analog is directly fused to the N-terminal residue of a second peptide linker, and the C-terminal residue of the second peptide linker is directly fused to the N-terminal residue of the human IgG Fc region. Each monomer of BIF has the amino acid sequence set forth in SEQ ID NO:1:

FVNQHLCGSHLVEALELVCGERGFHYGGGGGGSGGGGGIVEQCCTSTCSL
DQLENYCGGGGGQGGGGQGGGGQGGGGGECPPCPAPPVAGPSVFLFPPKP
KDTLMISRTPEVTCVVVDVSHEDPEVQFNWYVDGVEVHNAKTKPREEQFN
STFRVVSVLTVVHQDWLNGKEYKCKVSNKGLPAPIEKTISKTKGQPREPQ
VYTLPPSREEMTKNQVSLTCLVKGFYPSDIAVEWESNGQPENNYKTTPPM
LDSDGSFFLYSKLTVDKSRWQQGNVFSCSVMHEALHNHYTQKSLSLSPG

(SEQ ID NO:1). Each monomer includes intrachain disulfide bonds between cysteine residues at positions 7 and 44, 19 and 57, 43 and 48, 114 and 174 and 220 and 278. The two monomers are attached by disulfide bonds between the cysteine residues at positions 80 and 83 to form the dimer. The structure, function and production of BIF are described in more detail in U.S. Pat. No. 9,855,318.

When used herein, the term “BIF” refers to any insulin receptor agonist comprised of two monomers having the amino acid sequence of SEQ ID NO: 1, including any protein that is the subject of a regulatory submission seeking approval of an insulin receptor agonist product that relies in whole or part upon data submitted to a regulatory agency by Eli Lilly and Company relating to BIF, regardless of whether the party seeking approval of said product actually identifies the insulin receptor agonist as BIF or uses some other term.

The concentration of insulin-Fc fusion in compositions of the present invention must be sufficient to allow for administration of the range of insulin doses needed by patients having T2DM and TIDM with a broad range of insulin requirements. Currently available basal insulin products suitable for once-daily dosing, such as LANTUS (insulin glargine), TOUJEO (insulin glargine), TRESIBA (insulin degludec) and LEVEMIR (insulin detemir) are available in concentrations ranging from 100 insulin units (IU)/mL to 300 IU/mL. In certain embodiments of the present invention, the insulin-Fc fusion is present in concentrations ranging from about 100 to about 2000 insulin units (IU)/mL. In certain embodiments, the insulin-Fc fusion is present in a concentration of about 250 IU/mL, 500 IU/mL or 1000 IU/mL. The concentration of insulin-Fc fusion may also be expressed as mass per volume. For example, in certain embodiments wherein the insulin-Fc fusion is BIF, the concentration of BIF is between about 5-30 mg/mL. In certain embodiments, the concentration of BIF is selected from the group consisting of 7.15, 14.3 and 28.6 mg/mL.

The formulations of the present invention are sterile when first produced, however, when the composition is provided in a multi-use vial or cartridge, anti-microbial preservatives that are compatible with the insulin-Fc fusion and any other components of the formulation are added at sufficient strength to meet regulatory and pharmacopeial anti-microbial preservative requirements for multi-use products. These requirements include tests designed to challenge the ability of preservative to inhibit or kill microorganisms that may be inadvertently introduced into the product. Guidance for performing these tests is provided in the United States Pharmacopeia (USP)<51>“Antimicrobial Effectiveness Testing,” and the European Pharmacopeia (Ph. Eur. Or EP) 5.1.3 “Efficacy of Antimicrobial Preservation.” See. e.g., Meyer, B. D., et al., Antimicrobial preservative use in parenteral products: Past and present. JOURNAL OF PHARMACEUTICAL SCIENCES 2007, 96, (12), 3155-3167; Moser, C. L., Meyer, B. K., Comparison of compendial antimicrobial effectiveness tests: A review. AAPS PHARM. SCI. TECH 2011, 12, (1), 222-226.

The acceptance criteria referenced above evaluate the logio reduction of microbial counts at various defined timepoints and compare those counts to the initial time zero inoculum levels. For Example, USP criteria require not less than a 1.0-log reduction from the initial bacterial count at 7 days, not less than a 3.0-log reduction from the initial count at 14 days, and no increase from the 14-day count at 28 days. The EP B criteria are considered mandatory by EU regulatory agencies and require at least a 1 log reduction of the initial bacterial count at 24 hours and a 3-log reduction at 7 days. As it is more stringent than the USP criteria, any formulation that meets the EP B criteria would also meet the USP <51>criteria. The EP A criteria are the most stringent, requiring a 2-log reduction at 6 hours and 3-log reduction at 24 hours. The EP A criteria are difficult to achieve with many preservative systems, and often the preservative added to achieve EP A has detrimental effects on the product and/or is at toxic levels to patients are considered more achievable.

Therapeutic insulin products currently available for subcutaneous administration are multi-use products, and thus must meet regulatory requirements for anti-microbial effectiveness, including the USP and EP B criteria. Preservatives commonly used to meet those requirements include phenol (CAS No. 108-95-2, molecular formula C₆H₅₀H, molecular weight 94.11), and m-cresol (CAS No. 108-39-4, molecular formula C₇H₈₀, molecular weight 108.14), as in the products listed below in Table 1.

TABLE 1
Examples of preservatives used in commercially
available insulin products.

| Product(s) | Preservative(s) | Concentration(s) | |
|---|---|---|---|
| APIDRA ® (insulin glulisine) | m-cresol | 3.15 | mg/mL |
| HUMALOG ® (insulin lispro) | | | |
| LYUMJEV ™ (insulin lispro-aabc) | | | |
| HUMULIN ® R (human insulin) | m-cresol | 2.5 | mg/mL |
| LANTUS ® (insulin glargine) | m-cresol | 2.7 | mg/mL |
| BASAGLAR ® (insulin glargine) | | | |
| FIASP ® (insulin aspart) | m-cresol | 1.72 | mg/mL |
| NOVOLOG ® (insulin aspart) | phenol | 1.50 | mg/mL |
| TRESIBA ® (insulin degludec) | | | |
| LEVEMIR ® (insulin detemir) | m-cresol | 2.06 | mg |
| | phenol | 1.80 | mg |

In formulations of insulin-Fc fusions, like BIF, however, m-cresol and phenol in those concentrations result in precipitation of the protein, and thus cannot be used to provide sufficient antimicrobial efficacy to meet USP and EP requirements. The formulations of the present invention, therefore, rely on the use of different preservatives: phenoxyethanol (CAS No. 122-99-6, molecular formula C8H1002, molecular weight 138.16 g/mol) and/or benzyl alcohol (CAS No. 100-51-6, molecular formula C₇H₈O, molecular weight 108.14 g/mol). Specifically, it has been found that anti-microbial effectiveness criteria may be met in formulations within the desired pH range of BIF, without causing unacceptable loss of physical stability, through the use of certain concentrations of phenol in combination with benzyl alcohol and/or phenoxyethanol.

The concentrations of phenol and benzyl alcohol and/or phenoxyethanol in formulations of the present invention must be sufficient to ensure the formulation meets minimum sterility requirements for parenteral products set forth in the USP and EP B guidance documents. When used herein, the term “sterile” refers to a formulation that meets those minimum sterility requirements.

The concentrations of these preservatives, however, must not be so high as to cause unacceptable physical or chemical stability issues with the insulin-Fc fusion protein. The compositions of the present invention are sufficiently stable to allow for storage and multiple weeks of use (referred to herein as the “in-use” period) without unacceptable loss of stability. In certain embodiments, the compositions are sufficiently stable to allow for an in-use period of at least 12 weeks. In certain embodiments, the compositions are sufficiently stable to allow for an in-use period of 12 weeks under refrigeration with 2 weeks 30° C. In certain embodiments, the compositions are sufficiently stable to allow for an in-use period of 8 weeks at 25° C. In certain embodiments, the compositions are sufficiently stable to allow for an in-use period of 12 weeks at 25° C. In certain embodiments, the compositions are sufficiently stable to allow for an in-use period of 8 weeks at 30° C. In certain embodiments, the compositions are sufficiently stable to allow for an in-use period of 12 weeks at 30° C.

With respect to phenol, some multi-dose parenteral drug products use 5 mg/ml phenol as preservative, but that concentration was found to result in protein precipitation in BIF formulations, so the concentration must be less than 5 mg/mL. The concentration of phenol in certain embodiments of the present invention ranges from 1.5 to 4 mg/mL. The concentration of phenol in certain embodiments of the present invention is about 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9 or 4.0 mg/mL. In certain preferred embodiments, the concentration of phenol ranges from 1.8 to 3.5 mg/mL. The concentration of phenol in certain preferred embodiments is about 1.8, 1.9, 2.0, 2.1, 2.2, 2.3, 2.4, 2.5, 2.6, 2.7, 2.8, 2.9, 3.0, 3.1, 3.2, 3.3, 3.4 or 3.5 mg/mL. In certain preferred embodiments, the concentration of phenol is about 1.8, 2, 2.5, 3 or 3.5 m/mL. In particularly preferred embodiments, the concentration of phenol is about 1.8 mg/mL. It should be noted that due to its physical properties phenol is typically added to aqueous compositions, such as those described herein, in the form of a 90% solution in water. For example, in many of the studies described below; phenol was added as “Phenol, liquefied, distilled,” which is 90% phenol with 10% water. In those studies, the phenol concentration listed refers to the concentration of the 90% solution added to the composition. Thus, the absolute phenol content in a composition prepared with 2 mg/mL of a 90% phenol solution would be 1.8 mg/mL. Unless stated otherwise, e.g., as in the studies described below as using 90% phenol solution, the concentration of phenol comprised in compositions of the present invention refers to the absolute phenol content.

The concentration of either phenoxyethanol or benzyl alcohol in the formulations of the present invention depends on the concentration of phenol, but must be present in sufficient concentrations that the formulation is sterile at the desired pH. For example, in certain embodiments at pH 6.5, 9 mg/mL phenoxyethanol is not sufficient to pass EP B criteria in the absence of phenol, but concentrations as low as 4 mg/mL may be used to pass EP B criteria when combined with phenol concentrations as low as 1.8 mg/mL. Similarly, in certain embodiments, 9 mg/mL benzyl alcohol is not sufficient to pass even USP criteria, but concentrations as low as 5 mg/mL pass USP criteria when used in combination with 1.8 mg/mL phenol.

In certain embodiments, the concentration of phenoxyethanol ranges from 4 mg/mL to 14 mg/mL. In certain embodiments, the concentration of phenoxyethanol is about 4, 5, 6, 7, 8, 9, 10, 11, 12, 13 or 14 mg/mL. In certain preferred embodiments, the concentration of phenoxyethanol is about 4 or about 8 mg/mL.

In certain embodiments, the concentration of benzyl alcohol ranges from 5 to 10 mg/mL. In certain embodiments, the concentration of benzyl alcohol is about 5, 6, 7, 8, 9 or 10 mg/mL. In certain preferred embodiments, the concentration of benzyl alcohol is about 9 mg/mL.

The pH of formulations of the present invention ranges from 5.5 to 7.5, In certain embodiments the pH is about 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4 or 7.5. In certain embodiments, the pH ranges from 6 to 7. In certain embodiments the pH is about 6.0, 6.1, 6.2, 6.3, 6.4, 6.5, 6.6, 6.7, 6.8, 6.9 or 7.0. Preferably, the pH of formulations of the present invention is at least at the PI of the insulin-Fc fusion. For formulations comprising BIF, the pH is preferably at least about 6.1. In certain embodiments comprising BIF, the pH ranges from 6.2 to 7.4. In certain embodiments comprising BIF, the pH ranges from 6.2 to 6.9. In certain embodiments comprising BIF, the pH ranges from 6.3 to 6.8. In a particularly preferred embodiment comprising BIF, the pH is about 6.5.

If desired a buffering agent may be included. Examples of such buffering agents are phosphates, such as dibasic sodium phosphate, citrate, sodium acetate and tris(hydroxymethyl)aminomethane, or TRIS. If a buffering compound is necessary, citrate or phosphate buffers are preferred. In certain embodiments, compositions of the present invention include a citrate buffer in a concentration ranging from 5 to 10 mM. In certain embodiments, compositions of the present invention include phosphate in a concentration ranging from 5 to 10 mM. In certain preferred embodiments, compositions of the present invention include phosphate in a concentration of about 5, 6, 7, 8, 9 or 10 mM. In certain preferred embodiments, compositions of the present invention include phosphate in a concentration of either about 5 or about 10 mM.

It is desirable to approximately match the tonicity (i.e., osmolality) of body fluids at the injection site as closely as possible when administering the compositions because solutions that are not approximately isotonic with body fluids can produce a painful stinging sensation when administered. Thus, it is desirable that the compositions be approximately isotonic with body fluids at the sites of injection. If the osmolality of a composition in the absence of a tonicity agent is sufficiently less than the osmolality of the tissue (for blood, about 300 mOsmol/kg: the European Pharmacopeial requirement for osmolality is >240 mOsmol/kg), then a tonicity agent should generally be added to raise the tonicity of the composition to about 300 mOsmol/kg. The osmolality of the composition is determined by the identities and concentrations of other excipients in the composition, including the stabilizing agent(s). Thus, the concentrations of all of the various excipients in a composition must be assessed in order to determine whether a tonicity agent must be added, and such assessments and determinations are readily made using standard techniques. See Remington: The Science and Practice of Pharmacy, David B. Troy and Paul Beringer, eds., Lippincott Williams & Wilkins, 2006, pp. 257-259: Remington: Essentials of Pharmaceutics, Linda Ed Felton, Pharmaceutical Press, 2013, pp. 277-300. Typical tonicity agents include glycerol (glycerin), mannitol and sodium chloride. If the addition of a tonicity agent is required, glycerin is preferred. In certain embodiments the concentration of glycerol is from about 10 to about 50 mg/mL. In certain embodiments the concentration of glycerol is from about 15 to about 35 mg/mL. In certain embodiments the concentration of glycerol is selected from the group consisting of about 15, 17, 20, 21 and 35 mg/mL. In certain preferred embodiments, the concentration of glycerin is about 17 mg/mL.

The compositions of the present invention may also include other excipients, including stabilizing agents such as surfactants. Examples of surfactants disclosed for use in parenteral pharmaceutical compositions include polysorbates, such as polysorbate 20 (TWEENR 20) and polysorbate 80 (TWEEN 80), polyethylene glycols such as PEG 400, PEG 3000, TRITON™ X-100, polyethylene glycols such as polyoxyethylene (23) lauryl ether (CAS Number: 9002-92-0, sold under trade name BRIJR), alkoxylated fatty acids, such as MYRJ™, polypropylene glycols, block copolymers such as poloxamer 188 (CAS Number 9003 Nov. 6, sold under trade name PLURONICR F-68) and poloxamer 407 (PLURONICR F127), sorbitan alkyl esters (e.g., SPANR), polyethoxylated castor oil (e.g., KOLLIPHORR, CREMOPHORR) and trehalose and derivatives thereof, such as trehalose laurate ester.

In certain embodiments, the composition comprises a surfactant selected from the group consisting of polysorbate 20, polysorbate 80 and poloxamer 188. Most preferred is poloxamer 188. In certain embodiments, the concentration of surfactant ranges from 0.01 to 10 mg/mL or 0.1 to 0.5 mg/mL. In preferred embodiments wherein the surfactant is poloxamer 188, the concentration of poloxamer 188 is about 0.4 mg/mL.

In certain embodiments, compositions of the present invention are provided in an article of manufacture such as a multi-use vial, a cartridge, a re-usable pen injector, a disposable pen device, a pump device for continuous subcutaneous insulin infusion therapy or another container closure system for use in a pump device for continuous subcutaneous insulin infusion therapy. In certain embodiments, compositions are provided in re-usable pen injectors that may be used to provide variable doses of insulin that may be adjusted in particular increments. For example, in certain embodiments, such a pen injector comprises 1500 units of insulin and can be adjusted in 5-unit increments to deliver a dose of up to 400 units in a single injection. In other embodiments, such a pen injector comprises 3000 units of insulin and can be adjusted in 10-unit increments to deliver a dose of up to 800 units in a single injection.

As used herein, the term “about” is intended to refer to an acceptable degree of error for the amount or quantity indicated given the nature or precision of the measurements. For example, the degree of error can be indicated by the number of significant FIGURES provided for the measurement, as is understood in the art, and includes but is not limited to a variation of +/−1 in the most precise significant FIGURE reported for the amount or quantity. Typical exemplary degrees of error are within 20 percent (%), preferably within 10%, and more preferably within 5% of a given value or range of values. Numerical quantities given herein are approximate unless stated otherwise, meaning that the term “about” can be inferred when not expressly stated.

### EXAMPLES

### Conformational Stability in the Presence of Preservatives

Studies are conducted on the conformational stability of BIF when formulated with various phenolic preservatives. The compositions are set forth in Table 2 below.

TABLE 2
Control and sample compositions.

| | | | | | Benzyl | Solu- |
|---|---|---|---|---|---|---|
| | | BIF | m-Cresol | Phenol | alcohol | tion |
| Sample | Solvent | (mg/mL) | (mg/mL) | (mg/mL) | (mg/mL) | pH |
| 1 | Water | 2 | — | — | — | — |
| 2 | Water | 2 | 3.15 | — | — | ~6.5 |
| 3 | Water | — | 3.15 | — | — | — |
| 4 | Water | 2 | — | 5 | — | ~6.5 |
| 5 | Water | — | — | 5 | — | — |
| 6 | Water | 2 | — | — | 9 | ~6.5 |
| 7 | Water | — | — | — | 9 | — |

Extrinsic fluorescence measurements are performed to assess the conformational stability of BIF. Extrinsic fluorescent dyes such as 1-anilinonaphthalene-8-sulfonate (ANS) are minimally fluorescent in aqueous environment, but become highly fluorescent in a polar, organic solvents. These fluorescent dyes have been used to detect the exposure of hydrophobic patch(es) on protein surface(s) and provide information about protein folding and unfolding processes. See. e.g., Hawe, A., et al., Extrinsic fluorescent dyes as tools for protein characterization. PHARMACEUTICAL RESEARCH 2008. 25 (7), 1487-1499. The extrinsic fluorescence method is a plate-based method using Bis-ANS fluorescent probe to measure the surface hydrophobicity of proteins in solution. Fluorescence spectra are measured using a SpectraMax i3x multi-mode microplate reader (Molecular Devices. San Jose, USA). Samples are positioned in a black polypropylene 96-well corning half area flat plates. Approximately 100 μL of sample compositions containing 5 μM dye are transferred to each well and measured at 25° C. The excitation wavelength (2Ex) is 390 nm, and the emission spectrum is scanned from 420 nm to 600 nm with 2-nm steps.

Peak fluorescence signals for BIF-containing compositions are provided in Table 3 below.

TABLE 3
Peak ANS fluorescence intensity measurements.

| Sample | Peak fluorescence intensity (a.u.) |
|---|---|
| 1 | 24066400 |
| 2 | 33857500 |
| 4 | 33108600 |
| 6 | 27998820 |

As shown in Table 3, in the absence of any preservatives, some fluorescence is detected, indicating hydrophobic patch(es) on the surface of BIF even in its native, folded state. Once the preservatives are added, the fluorescence intensities increase. In the absence of BIF, Bis-ANS and preservatives do not produce any fluorescence signals. Therefore, the observed intensities are due to the interaction between the BIF and preservative molecules and resultant partial unfolding of the protein, which leads to the exposure of more hydrophobic patch(es).

Furthermore, the intensity of the fluorescence signals correlated with the hydrophobicity of the preservatives, with m-cresol being the most hydrophobic and producing the strongest signal, followed by phenol and benzyl alcohol. Benzyl alcohol, being the least hydrophobic among the three preservatives, induced the least perturbation to the BIF conformation.

### Physical Stability in the Presence of Preservatives Used in Currently Available Insulin Products

Formulations are prepared at pH 6.5 that contain 28.6 mg/mL BIF and concentrations of m-cresol and/or phenol that have been used in insulin products sold in multidose presentations. The formulations are filled into glass vials, stored at room temperature and tested by visual inspection. Results are provided in Table 4 below:

TABLE 4
Physical appearance observations as function of preservative(s).

| Formulation # | Preservative(s) | Appearance |
|---|---|---|
| 1 | 3.15 mg/mL m-cresol | precipitation |
| 2 | 5 mg/mL phenol | precipitation |
| 3 | 9 mg/mL benzyl alcohol and 3.5 | clear |
| | mg/mL phenol | |
| 4 | 14 mg/mL phenoxyethanol and | clear |
| | 3.5 mg/mL phenol | |

Formulations 1-2 each result in precipitation of BIF drug substance, indicating physical instability. Formulations 3 and 4 remain clear, indicating BIF drug substance remains physically stable.

### Stability as Function of pH

Biophysical developability/high-throughput profiling studies are conducted on 2 mg/mL formulations of BIF in different buffer matrices and pH conditions. The onset of melting temperature Tₘ (T_{m, Onset}) was measured using differential scanning calorimetry (DSC). T_{m, Onset} is the temperature at which a folded protein starts to lose its native conformation, i.e., the higher the T_{m, Onset}, the less susceptible a protein to denaturation. Results are provided in Table 5 below.

TABLE 5
DSC Tm, onset temperature as a function
of buffer, ionic strength, and pH.

| | | | | T_{m, Onset} |
|---|---|---|---|---|
| BIF (mg/mL) | Buffer | NaCl (mM) | pH | (° C.) |
| 2 | Citrate, 10 mM | 25 | 5.5 | 60.29 |
| | | | 6.0 | 63.58 |
| | | | 6.5 | 65.61 |
| | | | 7.0 | 66.32 |
| | | 150 | 5.5 | 59.59 |
| | | | 6.0 | 62.78 |
| | | | 6.5 | 64.76 |
| | | | 7.0 | 65.41 |
| | Phosphate, 10 mM | 25 | 6.0 | 62.86 |
| | | | 6.5 | 64.06 |
| | | | 7.0 | 66.39 |
| | | | 7.5 | 66.90 |
| | | 150 | 6.0 | 63.36 |
| | | | 6.5 | 64.42 |
| | | | 7.0 | 65.79 |
| | | | 7.5 | 66.11 |

As the solution pH increased from 5.5 to 7.5, there is a corresponding increase in T_{m, Onset}, regardless of the buffer type or the ionic strength.

Studies are also conducted on the colloidal stability of BIF with and without preservatives at pH conditions below and above its pl of ≈6.1. BIF drug substance prepared in citrate buffer is used for pH titration, using 1.5 N citric acid or 1 N NaOH.

Compositions are visually inspected for opalescence, which is considered a precursor to potential liquid-liquid phase separation. Raut, A. S.; Kalonia, D. S., Pharmaceutical perspective on opalescence and liquid-liquid phase separation in protein solutions. Molecular Pharmaceutics 2016, 13 (5), 1431-1444. Compositions both with and without preservatives appear opalescent as the pH approaches the drug substance pl and become clear at pH above the pl.

These studies show BIF favors a pH higher than its pl with respect to conformational and colloidal stability.

### Preservative Concentrations and Antimicrobial Efficacy

A study is designed to study formulations of BIF drug product comprising varying concentrations of phenoxyethanol and benzyl alcohol, with or without phenol, for antimicrobial efficacy. Materials used to prepare the compositions are identified in Table 7 below.

TABLE 7
Ingredient information

| Material | CAS # | Supplier |
|---|---|---|
| BIF drug substance | n/a | Eli Lilly |
| Glycerin, synthetic | 56-81-5 | Eli Lilly |
| Poloxamer 188 | 9003-11-6 | BASF |
| Phenoxyethanol | 122-99-6 | A & C American Chemicals |
| Benzyl alcohol | 100-51-6 | Avantor |
| Phenol, liquefied, distilled^{a} | 108-95-2 | Eli Lilly |

^{a}“Phenol, liquefied, distilled” is 90% phenol with 10% water.

Compositions containing BIF and mixtures of phenol and either phenoxyethanol or benzyl alcohol are prepared as described in Tables 8 and 9 below:

TABLE 8
BIF drug product containing phenoxyethanol and liquefied phenol.

| | Composition (mg/mL, or otherwise stated) | | | | | | |
|---|---|---|---|---|---|---|---|
| | | | Glyc- | Poloxamer | Preservative | | |
| # | BIF | Solvent | erin | 188 | Phenoxyethanol | Phenol | pH |
| 1 | 45 | 5 mM | 35 | 0.4 | 14 | — | 6.5 |
| | | citrate | | | | | |
| 2 | 45 | 5 mM | 35 | 0.4 | 13 | — | 6.5 |
| | | citrate | | | | | |
| 3 | 45 | 5 mM | 35 | 0.4 | 12 | — | 6.5 |
| | | citrate | | | | | |
| 4 | 45 | 5 mM | 35 | 0.4 | 11 | — | 6.5 |
| | | citrate | | | | | |
| 5 | 45 | 5 mM | 35 | 0.4 | 10 | — | 6.5 |
| | | citrate | | | | | |
| 6 | 45 | 5 mM | 35 | 0.4 | 9 | — | 6.5 |
| | | citrate | | | | | |
| 7 | 45 | 5 mM | 35 | 0.4 | 4 | 3.5 | 6.5 |
| | | citrate | | | | | |
| 8 | 45 | 5 mM | 35 | 0.4 | 4 | 2.5 | 6.5 |
| | | citrate | | | | | |
| 9 | 45 | 5 mM | 35 | 0.4 | 4 | 2 | 6.5 |
| | | citrate | | | | | |
| 10 | 45 | 5 mM | 35 | 0.4 | 4 | 1.5 | 6.5 |
| | | citrate | | | | | |
| 11 | 45 | 5 mM | 35 | 0.4 | 4 | 1 | 6.5 |
| | | citrate | | | | | |
| 12 | 30 | Water | 20 | 0.4 | 4 | 3 | 7.5 |
| 13 | 30 | Water | 20 | 0.4 | 4 | 3 | 7 |

TABLE 9
BIF drug product containing benzyl alcohol and liquefied phenol

| | Composition (mg/mL, or otherwise stated) | | | | | | |
|---|---|---|---|---|---|---|---|
| | | | | | Preservative | | |
| | | | | Poloxamer | Benzyl | | |
| # | BIF | Solvent | Glycerin | 188 | alcohol | Phenol | pH |
| 14 | 30 | Water | 20 | 0.4 | 9 | — | 7.5 |
| 15 | 30 | Water | 20 | 0.4 | 9 | — | 7 |
| 16 | 30 | Water | 20 | 0.4 | 9 | — | 6.5 |
| 17 | 30 | Water | 20 | 0.4 | 9 | 3.5 | 7.5 |
| 18 | 30 | Water | 20 | 0.4 | 9 | 3 | 7.5 |
| 19 | 30 | Water | 20 | 0.4 | 9 | 2.5 | 7.5 |
| 20 | 30 | Water | 20 | 0.4 | 9 | 2 | 7.5 |
| 21 | 30 | Water | 20 | 0.4 | 7 | 2 | 7.5 |
| 22 | 30 | Water | 20 | 0.4 | 5 | 2 | 7.5 |
| 23 | 30 | Water | 14 | 0.4 | 8 | 2 | 7 |
| 24 | 30 | Water | 14 | 0.4 | 8 | 2 | 6.5 |

Approximately 150 mL of solutions are filtered through 0.22-μm PVDF filters and immediately transferred to sterilized glass containers. The samples are stored at 5° C. until antimicrobial efficacy test was performed.

AET is performed by inoculating the test solutions with pure cultures of various microorganisms to represent common potential microbial contaminants. Specifically, solutions are inoculated with the following microorganisms listed in USP <51> and EP 5.1.3: Aspergillus brasiliensis spores. Candida albicans, Escherichia coli, Pseudomonas aeruginosa and Staphylococcus aureus. The inoculated solutions are stored at controlled room temperature (20° C. to 25° C.) in refrigerated incubators. Viable cell concentrations in inoculated vials are determined by plate counts at initial, 6 hours, 24 hours, 7 days, 14 days, and 28 days after inoculation. The results are compared to the acceptance criteria set forth in EP 5.1.3 and USP <51>, and the formulations are determined to either pass or fail each test criterion. The EP “A” criteria are considered the most stringent, followed by the EP “B” criteria, and then the USP criteria. The objective of the present study is to identify formulations of BIF drug product that meet the EP B and USP criteria.

Results for phenoxyethanol containing compositions are provided in Table 10 below:

TABLE 10
AET results of BIF drug product containing
phenoxyethanol and liquefied phenol.

| | Phenoxyethanol | Phenol | | | |
|---|---|---|---|---|---|
| Sample # | (mg/mL) | (mg/mL) | pH | EP B | USP |
| 1 | 14 | — | 6.5 | Pass | Pass |
| 2 | 13 | — | 6.5 | Pass | Pass |
| 3 | 12 | — | 6.5 | Pass | Pass |
| 4 | 11 | — | 6.5 | Pass | Pass |
| 5 | 10 | — | 6.5 | Pass | Pass |
| 6 | 9 | — | 6.5 | Fail | Pass |
| 7 | 4 | 3.5 | 6.5 | Pass | Pass |
| 8 | 4 | 2.5 | 6.5 | Pass | Pass |
| 9 | 4 | 2 | 6.5 | Pass | Pass |
| 10 | 4 | 1.5 | 6.5 | Fail | Pass |
| 11 | 4 | 1 | 6.5 | Fail | Fail |
| 12 | 4 | 3 | 7.5 | Pass | Pass |
| 13 | 4 | 3 | 7.0 | Pass | Pass |

As shown in Table 10, phenoxyethanol by itself can be an effective preservative. At concentrations of 10 mg/mL or higher, EP B and USP criteria are met. In addition, combinations of 4 mg/mL phenoxyethanol and 2 mg/mL or higher of liquefied phenol are also able to meet EP B and USP criteria. Finally, the EP B and USP criteria are met across a range of pH.

Results for benzyl alcohol containing compositions are provided in Table 11 below.

TABLE 11
AET results of BIF drug product containing
benzyl alcohol and liquefied phenol.

| | Benzyl alcohol | Phenol | | | | |
|---|---|---|---|---|---|---|
| Formulation # | (mg/mL) | (mg/mL) | pH | EP A | EP B | USP |
| 14 | 9 | — | 7.5 | Fail | Fail | Fail |
| 15 | 9 | — | 7.0 | Fail | Fail | Fail |
| 16 | 9 | — | 6.5 | Fail | Fail | Fail |
| 17 | 9 | 3.5 | 7.5 | Pass | Pass | Pass |
| 18 | 9 | 3 | 7.5 | Pass | Pass | Pass |
| 19 | 9 | 2.5 | 7.5 | Pass | Pass | Pass |
| 20 | 9 | 2 | 7.5 | Pass | Pass | Pass |
| 21 | 7 | 2 | 7.5 | Fail | Pass | Pass |
| 22 | 5 | 2 | 7.5 | Fail | Fail | Pass |
| 23 | 8 | 2 | 7.0 | Fail | Pass | Pass |
| 24 | 8 | 2 | 6.5 | Fail | Pass | Pass |

As seen in Table 11, solutions containing benzyl alcohol at 9 mg/mL and no phenol fail to meet EP B and USP criteria, likely due to the fact that the pH range is above that considered optimal for benzyl alcohol. See Meyer, B. D., et al., Anti-microbial preservative use in parenteral products: Past and present. JOURNAL OF PHARMACEUTICAL SCIENCES 2007, 96, (12), 3155-3167. When combined with phenol, however, solutions across the pH range for BIF drug product meet USP criteria, and—with the exception of the formulation containing the lowest concentrations tested of benzyl alcohol and phenol—EP B criteria. In addition, surprisingly the formulations containing 9 mg/mL benzyl alcohol and varying concentrations of liquefied phenol meet the more stringent EP A criteria.

### Preservative Comparison-Chemical and Physical Stability

A study is designed to test physical and chemical stability of two BIF formulations containing a combination of phenol and either phenoxyethanol or benzyl alcohol. The drug substance and excipients used are listed below in Table 12.

TABLE 12
Ingredient information

| Material | CAS # | Supplier |
|---|---|---|
| BIF drug substance (120 mg/mL) | n/a | Eli Lilly |
| Sodium phosphate monobasic | 10049-21-5 | Eli Lilly |
| monohydrate | | |
| Sodium phosphate dibasic | 7782-85-6 | Eli Lilly |
| heptahydrate | | |
| Glycerin, synthetic | 56-81-5 | Eli Lilly |
| Poloxamer 188 | 9003-11-6 | BASF |
| Phenoxyethanol | 122-99-6 | A & C American |
| | | Chemicals |
| Benzyl alcohol | 100-51-6 | Avantor |
| Phenol, liquefied, distilled ^{a} | n/a | Eli Lilly |
| NaOH, 1N | n/a | Fisher Scientific |
| Water, purified | n/a | GE/Hospira |

^{a} “Phenol, liquefied, distilled (QA205HV1E)” is 90% phenol with 10% water.

Solutions are prepared comprising 50 mg/mL BIF and a buffer comprising a combination of sodium phosphate monobasic monohydrate and sodium phosphate dibasic heptahydrate to give a buffer strength of 5 mM phosphate and other components as indicated in Table 13 below.

TABLE 13
Compositions of BIF drug product containing phenol
and either phenoxyethanol or benzyl alcohol:

| | | Poloxamer | | Benzyl | | |
|---|---|---|---|---|---|---|
| | Glycerin | 188 | Phenoxyethanol | Alcohol | Phenol ^{a} | pH |
| 1 | 20 | 0.4 | 4 | — | 3 | 6.4 |
| 2 | 20 | 0.4 | 4 | — | 3 | 6.7 |
| 3 | 20 | 0.4 | 4 | — | 3 | 7.0 |
| 4 | 15 | 0.4 | — | 8 | 2 | 6.4 |
| 5 | 15 | 0.4 | — | 8 | 2 | 6.7 |
| 6 | 15 | 0.4 | — | 8 | 2 | 7.0 |

Purified water was used as solvent;
^{a} “Phenol” listed in the studies is “Phenol, liquefied, distilled”, which is 90% phenol with 10% water.
^{c} pH was adjusted to the target pH using 1N NaOH during compounding

Solutions are filtered through 0.22-μm PVDF filters and immediately transferred to sterilized glass containers and then filled into 5 mL glass vials. Vials are capped and stored at 5° C., 25° C., and 30° C. for up to six months. Samples are submitted for testing with various stability indicating assays at various time points.

Results are provided in Tables 14-19 below:

TABLE 14
pH of BIF drug product formulations.

| Temp. | Time | | | | | | |
|---|---|---|---|---|---|---|---|
| (° C.) | (month) | 1 | 2 | 3 | 4 | 5 | 6 |
| 5 | 0 | 6.4 | 6.6 | 6.9 | 6.4 | 6.6 | 6.9 |
| | 1 | 6.4 | 6.6 | 6.9 | 6.4 | 6.6 | 6.9 |
| | 2 | 6.4 | 6.6 | 6.9 | 6.4 | 6.6 | 6.9 |
| | 3 | 6.4 | 6.7 | 7.0 | 6.4 | 6.7 | 7.0 |
| | 6 | 6.4 | 6.6 | 6.9 | 6.4 | 6.6 | 6.9 |
| 25 | 0 | 6.4 | 6.6 | 6.9 | 6.4 | 6.6 | 6.9 |
| | 1 | 6.4 | 6.6 | 6.9 | 6.4 | 6.6 | 6.9 |
| | 2 | 6.4 | 6.6 | 6.9 | 6.4 | 6.6 | 6.9 |
| | 3 | 6.4 | 6.7 | 6.9 | 6.4 | 6.6 | 7.0 |
| | 6 | 6.4 | 6.6 | 6.9 | 6.4 | 6.6 | 6.9 |
| 30 | 0 | 6.4 | 6.6 | 6.9 | 6.4 | 6.6 | 6.9 |
| | 1 | 6.4 | 6.6 | 6.9 | 6.4 | 6.6 | 7.0 |
| | 2 | 6.4 | 6.6 | 6.9 | 6.4 | 6.6 | 6.9 |
| | 3 | 6.4 | 6.7 | 6.9 | 6.3 | 6.6 | 6.9 |

TABLE 15
BIF monomer and total aggregates by SEC.

| Temp. | Time | Monomer (%) | | | | | | Total aggregates (%) | | | | | |
|---|---|---|---|---|---|---|---|---|---|---|---|---|---|
| (° C.) | (month) | −1 | −2 | −3 | −4 | −5 | −6 | −1 | −2 | −3 | −4 | −5 | −6 |
| 5 | 0 | 98.5 | 98.2 | 98.0 | 98.5 | 98.2 | 97.8 | 1.5 | 1.7 | 2.0 | 1.5 | 1.7 | 2.2 |
| | 1 | 98.5 | 98.2 | 98.0 | 98.5 | 98.2 | 97.8 | 1.5 | 1.8 | 2.0 | 1.5 | 1.8 | 2.2 |
| | 2 | 98.2 | 98.2 | 97.9 | 98.3 | 98.2 | 97.8 | 1.8 | 1.8 | 2.1 | 1.7 | 1.8 | 2.2 |
| | 3 | 98.3 | 98.2 | 97.9 | 98.3 | 98.1 | 97.8 | 1.7 | 1.8 | 2.0 | 1.7 | 1.9 | 2.2 |
| | 6 | 98.2 | 98.0 | 97.9 | 98.2 | 98.0 | 97.6 | 1.8 | 2.0 | 2.1 | 1.8 | 2.0 | 2.4 |
| 25 | 0 | 98.5 | 98.2 | 98.0 | 98.5 | 98.2 | 97.8 | 1.5 | 1.7 | 2.0 | 1.5 | 1.7 | 2.2 |
| | 1 | 98.1 | 98.0 | 97.9 | 98.1 | 98.0 | 97.7 | 1.9 | 2.0 | 2.1 | 1.9 | 2.0 | 2.2 |
| | 2 | 98.7 | 97.7 | 97.6 | 97.7 | 97.7 | 97.5 | 2.2 | 2.3 | 2.3 | 2.2 | 2.3 | 2.5 |
| | 3 | 98.6 | 97.6 | 97.4 | 97.6 | 97.5 | 97.3 | 2.3 | 2.4 | 2.4 | 2.4 | 2.4 | 2.5 |
| | 6 | 98.9 | 97.1 | 97.0 | 97.0 | 96.9 | 96.9 | 3.0 | 2.8 | 2.8 | 2.9 | 3.0 | 2.9 |
| 30 | 0 | 98.5 | 98.2 | 98.0 | 98.5 | 98.2 | 97.8 | 1.5 | 1.7 | 2.0 | 1.5 | 1.7 | 2.2 |
| | 1 | 98.8 | 97.8 | 97.6 | 97.9 | 97.7 | 97.5 | 2.2 | 2.2 | 2.3 | 2.1 | 2.2 | 2.4 |
| | 2 | 98.3 | 97.1 | 97.2 | 97.3 | 97.1 | 97.0 | 2.6 | 2.7 | 2.7 | 2.6 | 2.8 | 2.9 |
| | 3 | 98.8 | 96.9 | 97.0 | 96.7 | 96.9 | 96.8 | 3.0 | 2.9 | 2.8 | 3.2 | 3.0 | 3.0 |

TABLE 16
Sub-visible particulate matter of BIF drug product formulations by HIAC.

| 1 month (part/mL) | | |
|---|---|---|
| 5° C. | 25° C. | 30° C. |

| Lot | ≥2 | ≥5 | ≥10 | ≥25 | ≥2 | ≥5 | ≥10 | ≥25 | ≥2 | ≥5 | ≥10 | ≥25 |
|---|---|---|---|---|---|---|---|---|---|---|---|---|
| # | μm | μm | μm | μm | μm | μm | μm | μm | μm | μm | μm | μm |
| 1 | 111 | 30 | 9 | 3 | 115 | 29 | 8 | 1 | 297 | 81 | 24 | 1 |
| 2 | 160 | 37 | 9 | 0 | 167 | 37 | 11 | 1 | 135 | 38 | 7 | 0 |
| 3 | 195 | 66 | 20 | 1 | 239 | 83 | 25 | 3 | 154 | 36 | 9 | 0 |
| 4 | 172 | 49 | 21 | 1 | 306 | 86 | 26 | 3 | 171 | 53 | 19 | 2 |
| 5 | 193 | 63 | 14 | 1 | 141 | 46 | 7 | 1 | 163 | 40 | 9 | 1 |
| 6 | 162 | 55 | 13 | 2 | 111 | 35 | 9 | 1 | 91 | 30 | 7 | 1 |

| 2 months (part/mL) | | |
|---|---|---|
| 5° C. | 25° C. | 30° C. |

| Lot | ≥2 | ≥5 | ≥10 | ≥25 | ≥2 | ≥5 | ≥10 | ≥25 | ≥2 | ≥5 | ≥10 | ≥25 |
|---|---|---|---|---|---|---|---|---|---|---|---|---|
| # | μm | μm | μm | μm | μm | μm | μm | μm | μm | μm | μm | μm |
| 1 | 747 | 273 | 87 | 8 | 282 | 62 | 12 | 1 | 389 | 112 | 31 | 3 |
| 2 | 144 | 46 | 9 | 1 | 190 | 53 | 14 | 1 | 248 | 69 | 18 | 1 |
| 3 | 297 | 103 | 50 | 19 | 171 | 52 | 13 | 0 | 305 | 101 | 35 | 9 |
| 4 | 418 | 112 | 44 | 7 | 91 | 34 | 15 | 1 | 292 | 67 | 16 | 0 |
| 5 | 227 | 65 | 25 | 5 | 104 | 30 | 9 | 3 | 356 | 97 | 28 | 4 |
| 6 | 158 | 64 | 29 | 9 | 141 | 45 | 13 | 2 | 139 | 48 | 11 | 0 |

| | 3 months (part/mL) | | | | | | | | | | | |
|---|---|---|---|---|---|---|---|---|---|---|---|---|
| | 5° C. | | | | 25° C. | | | | 30° C. | | | |
| Lot | ≥2 | ≥5 | ≥10 | ≥25 | ≥2 | ≥5 | ≥10 | ≥25 | ≥2 | ≥5 | ≥10 | ≥25 |
| # | μm | μm | μm | μm | μm | μm | μm | μm | μm | μm | μm | μm |
| 1 | 158 | 47 | 14 | 3 | 281 | 67 | 26 | 6 | 605 | 145 | 35 | 5 |
| 2 | 156 | 47 | 22 | 6 | 241 | 101 | 48 | 21 | 285 | 87 | 39 | 12 |
| 3 | 147 | 31 | 4 | 1 | 215 | 60 | 19 | 4 | 195 | 61 | 19 | 5 |
| 4 | 469 | 144 | 54 | 6 | 369 | 113 | 57 | 15 | 499 | 180 | 87 | 42 |
| 5 | 210 | 87 | 51 | 23 | 151 | 40 | 11 | 0 | 375 | 114 | 48 | 11 |
| 6 | 138 | 77 | 56 | 23 | 185 | 62 | 24 | 6 | 246 | 94 | 49 | 16 |

TABLE 17
Sub-visible particulate matter by MFI.

| | 1 month (part/mL) | | | | | | | | | | | |
|---|---|---|---|---|---|---|---|---|---|---|---|---|
| | 5° C. | | | | 25° C. | | | | 30° C. | | | |
| | | | ≥5 | | | | ≥5 | | | | ≥5 | |
| Lot | >2 | ≥5 | μm & >0.85 | | >2 | >5 | μm& >0.85 | | >2 | >5 | μm& >0.85 | |
| # | μm | μm | AR | CF | μm | μm | AR | CF | μm | μm | AR | CF |
| 1 | 550 | 108 | 23 | 0.22 | 648 | 42 | 35 | 0.84 | 1246 | 113 | 37 | 0.32 |
| 2 | 233 | 52 | 13 | 0.26 | 242 | 63 | 18 | 0.29 | 232 | 52 | 23 | 0.45 |
| 3 | 400 | 118 | 37 | 0.31 | 340 | 67 | 48 | 0.73 | 223 | 47 | 33 | 0.71 |
| 4 | 583 | 225 | 18 | 0.08 | 440 | 150 | 52 | 0.34 | 307 | 93 | 33 | 0.36 |
| 5 | 477 | 147 | 37 | 0.25 | 178 | 67 | 35 | 0.53 | 343 | 60 | 33 | 0.56 |
| 6 | 825 | 152 | 23 | 0.15 | 88 | 33 | 8 | 0.25 | 128 | 40 | 20 | 0.5 |
| | 2 months (part/mL) | | | | | | | | | | | |
| | 5° C. | | | | 25° C. | | | | 30° C. | | | |
| | | | ≥5 | | | | ≥5 | | | | ≥5 | |
| Lot | >2 | ≥5 | μm& >0.85 | | >2 | >5 | μm& >0.85 | | >2 | >5 | μm& >0.85 | |
| # | μm | μm | AR | CF | μm | μm | AR | CF | μm | μm | AR | CF |
| 1 | 475 | 82 | 20 | 0.24 | 731 | 158 | 83 | 0.53 | 1308 | 183 | 90 | 0.49 |
| 2 | 495 | 137 | 35 | 0.26 | 305 | 75 | 28 | 0.38 | 427 | 113 | 72 | 0.63 |
| 3 | 716 | 252 | 85 | 0.34 | 758 | 95 | 52 | 0.54 | 318 | 82 | 23 | 0.29 |
| 4 | 536 | 200 | 25 | 0.12 | 963 | 227 | 88 | 0.39 | 1415 | 213 | 77 | 0.36 |
| 5 | 490 | 193 | 32 | 0.16 | 297 | 45 | 32 | 0.7 | 367 | 127 | 37 | 0.29 |
| 6 | 48 | 13 | 5 | 0.38 | 175 | 52 | 17 | 0.32 | 190 | 67 | 10 | 0.15 |

| 3 months (part/mL) | | |
|---|---|---|
| 5° C. | 25° C. | 30° C. |

| | | | ≥5 | | | | ≥5 | | | | ≥5 | |
|---|---|---|---|---|---|---|---|---|---|---|---|---|
| Lot | >2 | ≥5 | μm& >0.85 | | >2 | >5 | μm& >0.85 | | >2 | >5 | μm& >0.85 | |
| # | μm | μm | AR | CF | μm | μm | AR | CF | μm | μm | AR | CF |
| 1 | 710 | 168 | 20 | 0.12 | 1328 | 63 | 35 | 0.55 | 1460 | 170 | 120 | 0.71 |
| 2 | 278 | 88 | 15 | 0.17 | 906 | 62 | 35 | 0.57 | 318 | 42 | 33 | 0.8 |
| 3 | 523 | 160 | 25 | 0.16 | 373 | 52 | 42 | 0.81 | 355 | 52 | 45 | 0.87 |
| 4 | 1511 | 615 | 48 | 0.08 | 708 | 97 | 37 | 0.38 | 750 | 100 | 45 | 0.45 |
| 5 | 402 | 130 | 10 | 0.08 | 378 | 65 | 40 | 0.62 | 357 | 38 | 25 | 0.65 |
| 6 | 117 | 37 | 5 | 0.14 | 310 | 48 | 22 | 0.45 | 330 | 58 | 40 | 0.69 |

CF = circular fraction.

TABLE 18
BIF main peak purity and total impurities by RP-HPLC.

| | | Time | BIF main peak purity (%) | | | | | | Total impurities (%) | | | | | |
|---|---|---|---|---|---|---|---|---|---|---|---|---|---|---|
| Temp. | | (month) | 1 | 2 | 3 | 4 | 5 | 6 | 1 | 2 | 3 | 4 | 5 | 6 |
| 5° | C. | 0 | 78.9 | 78.4 | 78.4 | 78.5 | 78.6 | 77.5 | 21.1 | 21.6 | 21.6 | 21.5 | 21.4 | 22.5 |
| | | 1 | 79.5 | 79.8 | 79.4 | 79.8 | 79.8 | 79.3 | 20.5 | 20.2 | 20.6 | 20.2 | 20.2 | 20.7 |
| | | 2 | 77.9 | 77.2 | 77.7 | 76.9 | 76.8 | 77.0 | 22.1 | 22.8 | 22.3 | 23.1 | 23.2 | 23.0 |
| | | 3 | 78.8 | 78.7 | 79.5 | 80.1 | 78.7 | 78.6 | 21.2 | 21.3 | 20.5 | 19.9 | 21.3 | 21.4 |
| | | 6 | 79.9 | 79.0 | 78.3 | 78.7 | 80.0 | 78.9 | 20.1 | 21.0 | 21.7 | 21.3 | 20.0 | 21.1 |
| 25° | C. | 0 | 78.9 | 78.4 | 78.4 | 78.5 | 78.6 | 77.5 | 21.1 | 21.6 | 21.6 | 21.5 | 21.4 | 22.5 |
| | | 1 | 80.2 | 79.7 | 79.1 | 79.9 | 79.6 | 79.4 | 19.8 | 20.3 | 20.9 | 20.1 | 20.4 | 20.6 |
| | | 2 | 75.6 | 75.4 | 72.4 | 74.4 | 72.4 | 72.4 | 24.4 | 24.6 | 27.6 | 25.6 | 27.6 | 27.6 |
| | | 3 | 76.8 | 75.9 | 71.7 | 76.4 | 74.5 | 72.8 | 23.2 | 24.1 | 28.3 | 23.6 | 25.5 | 27.2 |
| | | 6 | 74.7 | 70.7 | 67.2 | 73.4 | 71.2 | 67.0 | 25.3 | 29.3 | 32.8 | 26.6 | 28.8 | 33.0 |
| 30° | C. | 0 | 78.9 | 78.4 | 78.4 | 78.5 | 78.6 | 77.5 | 21.1 | 21.6 | 21.6 | 21.5 | 21.4 | 22.5 |
| | | 1 | 79.3 | 78.3 | 76.0 | 79.4 | 78.5 | 75.9 | 20.7 | 21.7 | 24.0 | 20.6 | 21.5 | 24.1 |
| | | 2 | 75.7 | 72.2 | 69.1 | 75.1 | 72.5 | 68.6 | 24.3 | 27.8 | 30.9 | 24.9 | 27.5 | 31.4 |
| | | 3 | 73.9 | 70.2 | 66.8 | 73.0 | 69.5 | 65.5 | 26.1 | 29.8 | 33.2 | 27.0 | 30.5 | 34.5 |

TABLE 19
BIF main peak, total acidic variants, and total basic variants by AEX.

| Temp. | Time | BIF main peak (%) | | | | | | Total acidic variants (TAV) (%) | | | | | | Total basic variants (TBV) (%) | | | | | |
|---|---|---|---|---|---|---|---|---|---|---|---|---|---|---|---|---|---|---|---|
| (° C.) | (month) | 1 | 2 | 3 | 4 | 5 | 6 | 1 | 2 | 3 | 4 | 5 | 6 | 1 | 2 | 3 | 4 | 5 | 6 |
| 5 | 0 | 65.1 | 65.1 | 65.0 | 65.1 | 65.2 | 65.1 | 32.3 | 32.3 | 32.4 | 32.3 | 32.2 | 32.3 | 2.6 | 2.6 | 2.6 | 2.6 | 2.6 | 2.6 |
| | 1 | 64.2 | 63.9 | 63.0 | 64.1 | 63.8 | 63.3 | 32.7 | 33.0 | 33.9 | 32.7 | 33.1 | 33.7 | 3.1 | 3.1 | 3.1 | 3.1 | 3.1 | 3.0 |
| | 2 | 62.2 | 61.1 | 60.8 | 62.1 | 61.7 | 61.0 | 34.7 | 35.1 | 36.1 | 34.8 | 35.2 | 35.9 | 3.2 | 3.1 | 3.1 | 3.1 | 3.2 | 3.1 |
| | 3 | 62.1 | 61.7 | 59.8 | 61.6 | 61.6 | 60.9 | 34.7 | 35.1 | 36.4 | 34.8 | 34.9 | 35.7 | 4.1 | 3.8 | 3.8 | 3.7 | 3.4 | 3.4 |
| | 6 | 60.7 | 59.6 | 57.2 | 59.7 | 59.7 | 58.1 | 34.7 | 35.8 | 38.1 | 35.0 | 36.2 | 37.7 | 4.4 | 4.4 | 4.5 | 3.9 | 3.9 | 4.0 |
| 25 | 0 | 65.1 | 65.1 | 65.0 | 65.1 | 65.2 | 65.1 | 32.3 | 32.3 | 32.4 | 32.3 | 32.2 | 32.3 | 2.6 | 2.6 | 2.6 | 2.6 | 2.6 | 2.6 |
| | 1 | 61.1 | 59.1 | 55.8 | 61.0 | 59.2 | 55.7 | 35.7 | 37.8 | 41.4 | 35.8 | 37.8 | 41.6 | 3.2 | 3 | 2.8 | 3.2 | 3.0 | 2.8 |
| | 2 | 55.5 | 52.2 | 46.8 | 55.6 | 52.1 | 46.6 | 41.3 | 44.9 | 50.6 | 41.3 | 45.0 | 50.8 | 3.2 | 2.9 | 2.6 | 3.1 | 2.9 | 2.6 |
| | 3 | 52.4 | 47.8 | 40.6 | 53.0 | 47.9 | 40.9 | 43.5 | 48.4 | 55.7 | 43.5 | 48.5 | 56.1 | 4.1 | 3.9 | 3.6 | 3.5 | 3.6 | 3.0 |
| | 6 | 42.7 | 36.0 | 26.4 | 43.5 | 36.2 | 26.4 | 52.2 | 59.2 | 69.4 | 52.2 | 59.8 | 69.9 | 4.9 | 4.6 | 3.9 | 4.2 | 3.8 | 3.4 |
| 30 | 0 | 65.1 | 65.1 | 65.0 | 65.1 | 65.2 | 65.1 | 32.3 | 32.3 | 32.4 | 32.3 | 32.2 | 32.3 | 2.6 | 2.6 | 2.6 | 2.6 | 2.6 | 2.6 |
| | 1 | 56.5 | 52.8 | 47.4 | 56.5 | 53.1 | 46.8 | 40.3 | 44.4 | 50.1 | 40.3 | 44.1 | 50.6 | 3.1 | 2.8 | 2.5 | 3.1 | 2.8 | 2.5 |
| | 2 | 46.9 | 40.7 | 33.0 | 47.0 | 40.9 | 32.2 | 50.2 | 56.7 | 64.9 | 50.1 | 56.6 | 65.7 | 3.0 | 2.5 | 2.1 | 2.9 | 2.5 | 2.1 |
| | 3 | 42.2 | 35.8 | 26.5 | 43.2 | 35.7 | 26.2 | 53.7 | 60.5 | 70.2 | 53.3 | 61.2 | 71.0 | 4.1 | 3.7 | 3.2 | 3.5 | 3.1 | 2.8 |

As shown in Table 14, solution pH remained constant throughout the study duration. Results from SEC and sub-visible particles also confirm the samples are physically stable at each pH, as shown in total aggregates (Table 15) and particulate matter by HIAC or MFI (Tables 16 and 17). The chemical stability of the formulations was assessed using RP-HPLC and AEX. The formulations show comparable chemical stability, as reflected in total impurities (Table 18) and total acidic variants (Table 19), and in this study were most stable at pH 6.4.

### Stability

A study is designed to evaluate the stability and functionality of multi-use BIF drug product in 3-mL cartridges. All compositions are made with 5 mM phosphate, 21 mg/mL glycerin and 0.4 mg/mL poloxamer. Other characteristics of the compositions are provided in Table 20 below.

TABLE 20
Solution characteristics of formulations tested.

| | | Benzyl | | | | |
|---|---|---|---|---|---|---|
| Compo- | BIF | alcohol | Phenol | | Density | Viscosity |
| sition ID | (mg/mL) | (mg/mL) | (mg/mL) ^{a} | pH | (g/cc) | (cPs) |
| A | 15 | — | — | 6.6 | 1.0081 | 1.203 |
| B | — | 9 | 2 | 6.6 | 1.0045 | 1.123 |
| 1 | 15 | 9 | 2 | 6.6 | 1.0089 | 1.244 |
| 2 | 15 | 9 | 2 | 6.3 | 1.0089 | 1.248 |
| 3 | 15 | 8.1 | 1.8 | 6.4 | 1.0088 | 1.243 |
| 4 | 15 | 9.9 | 2.2 | 6.4 | 1.0089 | 1.242 |
| 5 | 15 | 8.1 | 1.8 | 6.8 | 1.0089 | 1.238 |
| 6 | 15 | 9.9 | 2.2 | 6.8 | 1.0090 | 1.242 |

^{a} “Phenol” listed in the studies is “Phenol, liquefied, distilled”, which is 90% phenol with 10% water.

Compositions are filled in 3-mL glass cartridges and closed with siliconized chlorobutyl plungers and DNR-free disc seals. Cartridges are stored at 5° C., 25° C., 30° C. or 35° C. for up to 6 months. Samples are pulled for testing as indicated in Table 21. below.

TABLE 21
Analytical tests.

| Test | | | | | | Time point | | | | |
|---|---|---|---|---|---|---|---|---|---|---|
| method | Property | | Unit | Temp. | | 0 | 1 | 2 | 3 | 6 |
| SEC | Monomer | | % | 5° | C. | X | X | X | X | X |
| | Total aggregates | | | 25° | C. | | X | X | X | X |
| | Total fragments | | | 30° | C. | | X | X | X | — |
| | | | | 35° | C. | | X | X | X | — |
| RP-HPLC | main peak purity | | % | 5° | C. | X | X | X | X | X |
| | Total impurities | | | 25° | C. | | X | X | X | X |
| | | | | 30° | C. | | X | X | X | — |
| | | | | 35° | C. | | X | X | X | — |
| AEX | main peak purity | | % | 5° | C. | X | X | X | X | X |
| | Total acidic variants | | | 25° | C. | | X | X | X | X |
| | Total basic variants | | | 30° | C. | | X | X | X | — |
| | | | | 35° | C. | | X | X | X | — |
| Particulate | ≥2 | μm | Part/ | 5° | C. | X | X | X | X | X |
| matter by | ≥5 | μm | mL | 25° | C. | | X | X | X | X |
| HIAC | ≥10 | μm | | 30° | C. | | X | X | X | — |
| | ≥25 | μm | | 35° | C. | | X | X | X | — |
| Particulate | ≥2 | μm | Part/ | 5° | C. | X | X | X | X | X |
| matter by | ≥5 | μm | mL | 25° | C. | | X | X | X | X |

| | | | | | | | |
|---|---|---|---|---|---|---|---|
| MFI | ≥5 μm with aspect | 30° | C. | X | X | X | — |
| | ratio ≥0.85 | 35° | C. | X | X | X | — |
| | Circular fraction | | | | | | |

No obvious trends were observed for the HIAC or MFI data as related to pH. storage conditions or formulation compositions. Particle counts by HIAC are all within specifications for the ≥10 μm and ≥25 μm measurements.

Results for SEC. RP-HPLC and AEX are provided in Tables 22-24 below:

TABLE 22
Monomer and total aggregates by SEC.

| Temp. | | Monomer (%) | | | | | | | Total aggregates (%) | | | | | | |
|---|---|---|---|---|---|---|---|---|---|---|---|---|---|---|---|
| (° C.) | Month | −A | −1 | −2 | −3 | −4 | −5 | −6 | −A | −1 | −2 | −3 | −4 | −5 | −6 |
| 5 | 0 | 95.8 | 95.7 | 96.3 | 95.7 | 95.6 | 95.7 | 95.6 | 4.2 | 4.3 | 3.7 | 4.3 | 4.4 | 4.3 | 4.4 |
| | 1 | 95.9 | 95.8 | 96.1 | 95.8 | 95.7 | 95.8 | 95.8 | 4.1 | 4.2 | 3.9 | 4.2 | 4.3 | 4.2 | 4.2 |
| | 2 | 96.0 | 95.8 | 96.3 | 95.8 | 95.8 | 95.8 | 95.8 | 4.0 | 4.2 | 3.7 | 4.2 | 4.2 | 4.2 | 4.2 |
| | 3 | 95.9 | 95.8 | 95.9 | 95.8 | 95.8 | 92.3 | 95.9 | 4.1 | 4.2 | 4.1 | 4.2 | 4.2 | 7.7 | 4.1 |
| | 6 | 96.0 | 96.0 | 95.9 | 95.9 | 95.9 | 96.0 | 96.0 | 4.0 | 4.0 | 4.1 | 4.1 | 4.1 | 4.0 | 4.0 |
| 25 | 0 | 95.8 | 95.7 | 96.3 | 95.7 | 95.6 | 95.7 | 95.6 | 4.2 | 4.3 | 3.7 | 4.3 | 4.4 | 4.3 | 4.4 |
| | 1 | 96.3 | 96.2 | 96.1 | 96.1 | 96.1 | 96.3 | 96.3 | 3.7 | 3.8 | 3.9 | 3.9 | 3.9 | 3.7 | 3.7 |
| | 2 | 96.3 | 96.2 | 96.1 | 96.1 | 96.1 | 96.4 | 96.4 | 3.6 | 3.8 | 3.9 | 3.9 | 3.9 | 3.6 | 3.6 |
| | 3 | 96.3 | 96.2 | 96.0 | 96.1 | 96.0 | 96.4 | 96.4 | 3.6 | 3.8 | 4.0 | 3.9 | 3.9 | 3.6 | 3.6 |
| | 6 | 96.3 | 96.2 | 95.8 | 96.0 | 95.9 | 96.5 | 96.5 | 3.6 | 3.7 | 4.1 | 3.9 | 4.0 | 3.4 | 3.4 |
| 30 | 0 | 95.8 | 95.7 | 96.3 | 95.7 | 95.6 | 95.7 | 95.6 | 4.2 | 4.3 | 3.7 | 4.3 | 4.4 | 4.3 | 4.4 |
| | 1 | 96.4 | 96.3 | 96.1 | 96.2 | 96.1 | 96.4 | 96.4 | 3.6 | 3.7 | 3.8 | 3.8 | 3.8 | 3.6 | 3.3 |
| | 2 | 96.3 | 96.2 | 95.9 | 96.0 | 95.9 | 96.5 | 96.4 | 3.6 | 3.8 | 4.1 | 3.9 | 4.0 | 3.5 | 3.5 |
| | 3 | 96.2 | 96.1 | 95.8 | 95.9 | 95.9 | 96.5 | 96.4 | 3.7 | 3.8 | 4.1 | 4.0 | 4.0 | 3.4 | 3.4 |
| 35 | 0 | 95.8 | 95.7 | 96.3 | 95.7 | 95.6 | 95.7 | 95.6 | 4.2 | 4.3 | 3.7 | 4.3 | 4.4 | 4.3 | 4.4 |
| | 1 | 96.5 | 96.2 | 96.1 | 96.0 | 96.0 | 96.4 | 96.4 | 3.5 | 3.7 | 3.9 | 3.9 | 3.9 | 3.5 | 3.5 |
| | 2 | 96.5 | 96.1 | 96.0 | 95.9 | 95.9 | 96.6 | 96.4 | 3.4 | 3.8 | 3.9 | 4.0 | 4.0 | 3.3 | 3.5 |
| | 3 | 96.3 | 94.8 | 94.3 | 95.4 | 95.0 | 96.2 | 95.6 | 3.5 | 5.0 | 5.5 | 4.4 | 4.8 | 3.6 | 4.2 |

TABLE 23
Main peak purity and total impurities by RP-HPLC.

| Temp. | Time | Main peak purity (%) | | | | | | | | Total impurities (%) | | | | | | | |
|---|---|---|---|---|---|---|---|---|---|---|---|---|---|---|---|---|---|
| (° C.) | (month) | −A | −B | −1 | −2 | −3 | −4 | −5 | −6 | −A | −B | −1 | −2 | −3 | −4 | −5 | −6 |
| 5 | 0 | 78.6 | n/a | 78.3 | 78.0 | 78.1 | 78.0 | 77.9 | 78.0 | 21.4 | n/a | 21.7 | 22.0 | 21.9 | 22.0 | 22.1 | 22.0 |
| | 1 | 78.3 | n/a | 78.0 | 78.1 | 78.0 | 78.2 | 77.9 | 78.3 | 21.7 | n/a | 22.0 | 21.9 | 22.0 | 21.8 | 22.1 | 21.7 |
| | 2 | 75.6 | n/a | 76.4 | 76.4 | 76.8 | 75.8 | 76.7 | 76.1 | 24.4 | n/a | 23.6 | 23.6 | 23.2 | 24.2 | 23.3 | 23.9 |
| | 3 | 77.9 | n/a | 78.0 | 78.4 | 77.7 | 78.4 | 77.9 | 77.9 | 22.1 | n/a | 22.0 | 21.6 | 22.3 | 21.6 | 22.1 | 22.1 |
| | 6 | 74.0 | n/a | 74.5 | 74.0 | 74.1 | 74.7 | 72.4 | 73.5 | 26.0 | n/a | 25.5 | 26.0 | 25.9 | 25.3 | 27.6 | 26.5 |
| 25 | 0 | 78.6 | n/a | 78.3 | 78.0 | 78.1 | 78.0 | 77.9 | 78.0 | 21.4 | n/a | 21.7 | 22.0 | 21.9 | 22.0 | 22.1 | 22.0 |
| | 1 | 77.7 | n/a | 77.0 | 77.6 | 77.2 | 76.9 | 75.9 | 77.1 | 22.3 | n/a | 23.0 | 22.4 | 22.8 | 23.1 | 24.1 | 22.9 |
| | 2 | 72.1 | n/a | 74.7 | 75.1 | 74.4 | 74.8 | 73.4 | 72.7 | 27.9 | n/a | 25.3 | 24.9 | 25.6 | 25.2 | 26.6 | 27.3 |
| | 3 | 75.5 | n/a | 74.4 | 75.9 | 76.2 | 76.2 | 72.0 | 71.4 | 24.5 | n/a | 25.6 | 24.1 | 23.8 | 23.8 | 28.0 | 28.6 |
| | 6 | 70.0 | n/a | 66.6 | 68.9 | 68.6 | 68.8 | 64.2 | 64.6 | 30.0 | n/a | 33.4 | 31.1 | 31.4 | 31.2 | 35.8 | 35.4 |
| 30 | 0 | 78.6 | n/a | 78.3 | 78.0 | 78.1 | 78.0 | 77.9 | 78.0 | 21.4 | n/a | 21.7 | 22.0 | 21.9 | 22.0 | 22.1 | 22.0 |
| | 1 | 76.5 | n/a | 76.3 | 77.0 | 76.6 | 76.6 | 74.6 | 74.8 | 23.5 | n/a | 23.7 | 23.0 | 23.4 | 23.4 | 25.4 | 25.2 |
| | 2 | 70.4 | n/a | 71.9 | 72.9 | 73.2 | 72.5 | 69.6 | 69.8 | 29.6 | n/a | 28.1 | 27.1 | 26.8 | 27.5 | 30.4 | 30.2 |
| | 3 | 71.3 | n/a | 68.9 | 71.4 | 71.0 | 71.3 | 67.0 | 66.3 | 28.7 | n/a | 31.1 | 28.6 | 29.0 | 28.7 | 33.0 | 33.7 |
| 35 | 0 | 78.6 | n/a | 78.3 | 78.0 | 78.1 | 78.0 | 77.9 | 78.0 | 21.4 | n/a | 21.7 | 22.0 | 21.9 | 22.0 | 22.1 | 22.0 |
| | 1 | 73.9 | n/a | 73.1 | 74.8 | 74.6 | 74.5 | 71.8 | 71.5 | 26.1 | n/a | 26.9 | 25.2 | 25.4 | 25.5 | 28.2 | 28.5 |
| | 2 | 68.7 | n/a | 67.5 | 70.2 | 69.5 | 69.4 | 64.8 | 64.4 | 31.3 | n/a | 32.5 | 29.8 | 30.5 | 30.6 | 35.2 | 35.6 |
| | 3 | 66.0 | n/a | 62.3 | 68.2 | 66.1 | 65.9 | 58.5 | 58.4 | 34.0 | n/a | 37.7 | 31.8 | 33.9 | 34.1 | 41.5 | 41.6 |

TABLE 24
Main peak, total acidic varianta, and total basic variants by AEX.

| Temp. | | main peak (%) | | | | | | | Total acidic variants (TAV) (%) | | | | | | |
|---|---|---|---|---|---|---|---|---|---|---|---|---|---|---|---|
| (° C.) | Time | −A | −1 | −2 | −3 | −4 | −5 | −6 | −A | −1 | −2 | −3 | −4 | −5 | −6 |
| 5 | 0 | 61.5 | 60.4 | 60.5 | 60.7 | 60.6 | 60.6 | 60.3 | 35.3 | 33.1 | 32.7 | 33.1 | 32.5 | 33.2 | 32.9 |
| | 1 | 61.7 | 60.5 | 60.6 | 60.8 | 60.4 | 60.4 | 60.2 | 35.1 | 32.8 | 32.4 | 32.8 | 32.3 | 33.2 | 32.6 |
| | 2 | 60.6 | 59.9 | 60.1 | 60.7 | 59.6 | 59.6 | 59.1 | 36.9 | 35.2 | 34.8 | 34.6 | 35.2 | 35.8 | 35.8 |
| | 3 | 61.2 | 61.6 | 62.4 | 61.6 | 61.8 | 61.1 | 61.7 | 35.7 | 35.8 | 35 | 35.8 | 35.6 | 36.3 | 35.8 |
| | 6 | 64.4 | 61.7 | 63 | 62.6 | 62.7 | 61.5 | 61.4 | 33.2 | 32.5 | 31 | 31.9 | 31.1 | 33 | 32.5 |
| 25 | 0 | 61.5 | 60.4 | 60.5 | 60.7 | 60.6 | 60.6 | 60.3 | 35.3 | 33.1 | 32.7 | 33.1 | 32.5 | 33.2 | 32.9 |
| | 1 | 58 | 56.8 | 58.3 | 57.6 | 57.5 | 55.3 | 55 | 38.8 | 36.5 | 34.8 | 35.8 | 35.2 | 38.3 | 37.9 |
| | 2 | 54.3 | 51.7 | 55.3 | 54.7 | 53.9 | 49.6 | 49.7 | 43.2 | 43.3 | 39.4 | 40.4 | 40.7 | 45.8 | 45.1 |
| | 3 | 51.5 | 50.4 | 53.5 | 52.5 | 52.9 | 46.4 | 46.3 | 45.3 | 47.2 | 43.9 | 45 | 44.7 | 51.5 | 51.7 |
| | 6 | 45.8 | 40.4 | 47.1 | 45.4 | 44.7 | 34.9 | 34.5 | 51.8 | 54.1 | 46.9 | 49.2 | 49.1 | 60.1 | 59.8 |
| 30 | 0 | 61.5 | 60.4 | 60.5 | 60.7 | 60.6 | 60.6 | 60.3 | 35.3 | 33.1 | 32.7 | 33.1 | 32.5 | 33.2 | 32.9 |
| | 1 | 53.9 | 52.3 | 54.7 | 54.3 | 53.7 | 49.3 | 49 | 42.7 | 41 | 38.2 | 39.3 | 39 | 44.4 | 44 |
| | 2 | 46.4 | 43.9 | 49.2 | 48.4 | 48 | 39.8 | 39.3 | 51.1 | 51.1 | 45.4 | 46.7 | 46.5 | 55.7 | 55.6 |
| | 3 | 39.7 | 38.5 | 45.1 | 43.2 | 43.1 | 33.5 | 33.1 | 57.1 | 59.6 | 52.5 | 54.6 | 54.5 | 65 | 65.5 |
| 35 | 0 | 61.5 | 60.4 | 60.5 | 60.7 | 60.6 | 60.6 | 60.3 | 35.3 | 33.1 | 32.7 | 33.1 | 32.5 | 33.2 | 32.9 |
| | 1 | 47.3 | 44.8 | 49 | 47.9 | 47.4 | 41.1 | 39.8 | 49.5 | 48.5 | 43.8 | 45.7 | 45.3 | 52.8 | 53.2 |
| | 2 | 35.2 | 32.6 | 39.2 | 36.9 | 36.3 | 27 | 26.5 | 55.9 | 62.6 | 55.4 | 58.4 | 58.3 | 68.7 | 68.7 |
| | 3 | 26.3 | 24.1 | 32.4 | 29.5 | 29.1 | 18.4 | 17.8 | 70.6 | 74.7 | 65.9 | 68.9 | 69.5 | 80.7 | 81.4 |

| Temp. | Total basic variants (TBV) (%) | | | | | | |
|---|---|---|---|---|---|---|---|
| (° C.) | −A | −1 | −2 | −3 | −4 | −5 | −6 |
| 5 | 3.1 | 6.3 | 6.7 | 6.1 | 6.9 | 6.1 | 6.8 |
| | 3 | 6.6 | 6.9 | 6.3 | 7.1 | 6.3 | 7.1 |
| | 2.5 | 4.9 | 5.2 | 4.7 | 5.2 | 4.6 | 5.1 |
| | 3.1 | 2.6 | 2.6 | 2.6 | 2.6 | 2.6 | 2.5 |
| | 2.5 | 5.8 | 6 | 5.6 | 6.1 | 5.4 | 6.2 |
| 25 | 3.1 | 6.3 | 6.7 | 6.1 | 6.9 | 6.1 | 6.8 |
| | 3 | 6.6 | 6.8 | 6.4 | 7.2 | 6.3 | 7 |
| | 2.5 | 5 | 5.3 | 4.9 | 5.4 | 4.6 | 5.3 |
| | 3.3 | 2.3 | 2.6 | 2.5 | 2.4 | 2.1 | 2 |
| | 2.5 | 5.5 | 6 | 5.4 | 6.2 | 5 | 5.8 |
| 30 | 3.1 | 6.3 | 6.7 | 6.1 | 6.9 | 6.1 | 6.8 |
| | 3.2 | 6.5 | 7 | 6.5 | 7.2 | 6.2 | 6.9 |
| | 2.5 | 5 | 5.4 | 4.9 | 5.6 | 4.5 | 5.2 |
| | 3.2 | 1.9 | 2.4 | 2.2 | 2.1 | 1.6 | 1.5 |
| 35 | 3.1 | 6.3 | 6.7 | 6.1 | 6.9 | 6.1 | 6.8 |
| | 3 | 6.5 | 7.1 | 6.3 | 7.2 | 6 | 6.9 |
| | 2.4 | 4.8 | 5.4 | 4.8 | 5.4 | 4.3 | 4.8 |
| | 3 | 1.1 | 1.7 | 1.5 | 1.4 | 0.9 | 0.9 |

As seen in Table 22, all formulations were well behaved at 5° C., 25° C., and 30° C., with no noteworthy difference between the control and test samples, suggesting benzyl alcohol and phenol at studied concentrations did not induce significant protein denaturation Aggregate growth observed at 35° C. is largely driven by the thermal stress, as there is no clear trend among the samples.

As seen in Table 23, at 5° C., there is no difference between control and test samples. Differences can be seen at 25° C., 30° C., and 35° C. At these temperatures, the growth of total impurities increases as pH increases.

Similarly, as seen in Table 24, at 5° C., there is negligible growth in TAV. TAV growth is significant at elevated temperatures (25° C., 30° C., and 35° C.) and is correlated with increase in pH.

In summary, the preservatives tested showed minimal impact on the formulation stability, while the primary factors affecting stability were pH and temperature. At 5° C., the formulations remained stable with little growth in aggregation and chemical degradation, but as the storage temperatures increased (25° C., 30° C., and 35° C.), degradation accelerated correspondingly. Subvisible particulate matter were within the specifications for all study arms. Overall, the results from this study show robustness across the formulations tested.

### Chemical Stability as a Function of pH

Studies are designed to study the chemical stability of BIF formulations with and without preservatives at pH conditions above its pl.

Preservative-containing compositions are prepared are set forth below in Table 25.

TABLE 25
Compositions of BIF drug product.

| | Composition (mg/mL) ^{a} | | | | | | |
|---|---|---|---|---|---|---|---|
| | | Phosphate | | | | | |
| Lot | | buffer | Glyc- | Poloxamer | Benzyl | | |
| # | BIF | (mM) | erin | 188 | alcohol | Phenol ^{b} | pH ^{c} |
| 1 | 15 | 5 | 21 | 0.4 | 9 | 2 | 6.2 |
| 2 | 15 | 5 | 21 | 0.4 | 9 | 2 | 6.3 |
| 3 | 15 | 5 | 21 | 0.4 | 9 | 2 | 6.4 |
| 4 | 15 | 5 | 21 | 0.4 | 9 | 2 | 6.5 |
| 5 | 15 | 5 | 21 | 0.4 | 9 | 2 | 6.6 |
| 6 | 15 | 5 | 21 | 0.4 | 9 | 2 | 6.7 |
| 7 | 15 | 5 | 21 | 0.4 | 9 | 2 | 6.8 |
| 8 | 15 | 5 | 21 | 0.4 | 9 | 2 | 6.9 |

^{a} Purified water was used as solvent;
^{b} “Phenol” is “Phenol, liquefied, distilled”, which is 90% phenol with 10% water;
^{c} pH was adjusted to the target pH using 1N NaOH during sample preparation.

Compositions without preservatives are prepared as set forth below in Table 26.

TABLE 26
Compositions of BIF drug product.

| | Composition (mg/mL) ^{a} | | |
|---|---|---|---|
| Lot # | BIF | Phosphate buffer (mM) | pH ^{b} |
| 9 | 2 | 5 | 6.1 |
| 10 | 2 | 5 | 6.3 |
| 11 | 2 | 5 | 6.4 |
| 12 | 2 | 5 | 6.5 |
| 13 | 2 | 5 | 6.6 |
| 14 | 2 | 5 | 6.7 |
| 15 | 2 | 5 | 6.8 |
| 16 | 2 | 5 | 7.0 |

^{a} Purified water was used as solvent;
^{b} pH was adjusted to the target pH using 1N NaOH during sample preparation.

All solutions were filtered through 0.22-μm PVDF filters and immediately transferred to sterilized glass containers. In a laminar flow hood, the solutions were filled into glass vials. Vials were capped and stored at 5° C. and 30° C. for up to three months. At appropriate times, samples were withdrawn and submitted for testing.

Chemical stability is assessed by anion exchange chromatography (AEX). Results are provided in Tables 27 below.

TABLE 27
TAV in preservative-containing formulations as determined by AEX.

| Temp. | Time | Total acidic variants (TAV) (%) | | | | | | | |
|---|---|---|---|---|---|---|---|---|---|
| (° C.) | (month) | 1 | 2 | 3 | 4 | 5 | 6 | 7 | 8 |
| 5 | 0 | 37.3 | 37.1 | 37.1 | 37.4 | 37.2 | 36.2 | 36.7 | 36 |
| | 1 | 37.6 | 37.6 | 37.7 | 37.8 | 37.8 | 37.9 | 38.1 | 38.2 |
| | 2 | 37.4 | 37.6 | 37.7 | 37.5 | 37.7 | 37.6 | 38 | 38.4 |
| | 3 | 37.6 | 37.6 | 37.4 | 37.6 | 38 | 38.2 | 38 | 38.4 |
| 30 | 0 | 37.3 | 37.1 | 37.1 | 37.4 | 37.2 | 36.2 | 36 | 36 |
| | 1 | 40.7 | 41.2 | 41.9 | 42.3 | 43.3 | 44.2 | 45.1 | 45.9 |
| | 2 | 44.1 | 45.1 | 46.4 | 47.6 | 49 | 51.1 | 53.8 | 54.9 |
| | 3 | 48.2 | 49.5 | 51.1 | 53.4 | 55.6 | 58.1 | 60.7 | 63.3 |

TABLE 28
TAV in non-preserved formulations as determined by AEX.

| Temp. | Time | Total acidic variants (TAV) (%) | | | | | | | |
|---|---|---|---|---|---|---|---|---|---|
| (° C.) | (month) | 9 | 10 | 11 | 12 | 13 | 14 | 15 | 16 |
| 5 | 0 | 34.8 | 35.3 | 34.9 | 35.2 | 35.3 | 35.2 | 35.1 | 35.4 |
| | 1 | 34.3 | 34.3 | 35 | 34.4 | 34.6 | 34.8 | 35.2 | 35.1 |
| | 2 | 35.4 | 35.5 | 35.3 | 35.9 | 35.7 | 35.7 | 36.2 | 36.2 |
| | 3 | 34.6 | 34.8 | 34.8 | 34.5 | 35.1 | 35.4 | 35.7 | 36 |
| 30 | 0 | 34.8 | 35.3 | 34.9 | 35.2 | 35.3 | 35.2 | 35.1 | 35.4 |
| | 1 | 37.4 | 38.4 | 38.8 | 37.6 | 41 | 42.6 | 43.5 | 46.1 |
| | 2 | 73.4 | 43.9 | 45.8 | 46.9 | 48.7 | 51.1 | 52.6 | 55.9 |
| | 3 | 45.5 | 49.1 | 51.4 | 51.7 | 53.8 | 58.1 | 60.4 | 64.3 |

Table 28. TAV in non-preserved formulations as determined by AEX.

As seen in Tables 27-28, at 5° C., there is negligible growth in total acidic variants (TAV), while at 30° C. growth occurs in a pH-sensitive manner. The presence of preservatives in these compositions did not materially impact stability.

### Shelf-Life and In-Use

TAV is considered the most relevant chemical stability-indicating assay for BIF, so the results described above in Tables 27-28 are used for shelf life and in-use estimation. The following equation was used to factor out the accelerating effect of the storage temperature (T) at timepoint (1) to collapse the time scale to a single arbitrary reference temperature (T_{Ref}).

$${e}^{\frac{- {E}_{a}}{R} ( \frac{1}{T} - \frac{1}{{T}_{Ref}} )} \cdot t = {t}_{Ref}$$

An apparent activation energy (Eₐ) value of 21.5 kcal/mol was used, with a reference temperature of 5° C. Raut, A. S.: Kalonia, D. S., Pharmaceutical perspective on opalescence and liquid-liquid phase separation in protein solutions. Molecular Pharmaceutics 2016, 13 (5), 1431-1444. The validity of the assumption that Eₐ is approximately 21.5 kcal/mol is assessed empirically by graphing the analytical observations against time, or scaled to 5° C. with Eₐ=21.5 kcal/mol. If the true Eₐ is different than the assumed value, a consistent trend at one of the temperatures will arise, i.e., evidence that the assumed Eₐ does not adequately account for the temperature impact and the true Eₐ is different than the current estimate. Current stability results do not indicate that the assumption of Eₐ being 21.5 kcal/mol is invalid. Thus, the data in Tables 27-28 above serve as a tool to determine long-term stability under refrigerated conditions. The equivalent number of months at 5° C. for each product shelf life with in-use condition are presented in Table 29.

TABLE 29
Relationship between shelf-life and
in-use conditions and time at 5° C.

| Product shelf-life and in-use | Equivalent number |
|---|---|
| condition | of months at 5° C. |
| 24 months at 5° C. | 24 months |
| 24 months at 5° C. plus 2 weeks at 30° C. | 35.4 months |
| 24 months at 5° C. plus 4 weeks at 30° C. | 46.8 months |

The data in Tables 27-28 show that the preferred drug product pH in such embodiments is approximately 6.5 or lower.

### Clinical Study

A clinical study in healthy participants is designed to compare acute injection-site pain intensity associated with matrices containing preservatives and tonicity agent. Each participant received one 0.6-mL SC injection on Day 1 in Periods 1 through 5. No active drug was administered. The 5 solution formulations were as follows:

TABLE 30
Formulation tested for injection-site pain.

| | Composition | | |
|---|---|---|---|
| | | Tonicity | |
| Formulation | Buffer | agent | Preservative |
| 1 | Unbuffered | Glycerol | Phenoxyethanol (0.4% w/v) + |
| | (Water) | | Phenol (0.3% w/v) |
| 2 | 5 mM Citrate | Glycerol | Phenoxyethanol (0.4% w/v) + |
| | | | Phenol (0.3% w/v) |
| 3 | Unbuffered | Glycerol | Benzyl alcohol (0.8% w/v) + |
| | (Water) | | Phenol (0.2% w/v) |
| 4 | 5 mM Citrate | Glycerol | Benzyl alcohol (0.8% w/v) + |
| | | | Phenol (0.2% w/v) |
| 5 | Unbuffered | Glycerol | None |
| | (Water) | | |

Injection-site pain was evaluated and quantified using a 100-mm visual analog scale (VAS), where 0 indicated “no pain” and 100 indicated “worst imaginable pain”. Data were listed and summarized by treatment and time point.

A mixed effects model was used to analyze the continuous injection-site pain from VAS pain scores at each time post injection for each formulation. The model was by time point of measurement after injections and included treatment (solution formulations), injection order within cohort (1^{st}, 2^{nd}, 3^{rd}, 4^{th}, or 5^{th} injection of the period), cohort (injection sequence group participants were randomized to) as fixed factors and participant as a random effect. The Kenward-Roger method was used to estimate the denominator degrees of freedom. Type III test for the least squares (LS) mean was used for statistical comparison: 95% confidence intervals (CI) for the difference were also reported. A difference in LS means was considered statistically significant if the 95% CI excluded zero.

All adverse events (AE) were listed. Treatment-emergent AEs were summarized. Any serious adverse events (SAE) were listed.

Injection-site reaction (ISR) questionnaires were collected at prespecified time points and for spontaneously reported ISRs. ISR data were listed and summarized by treatment in frequency tables.

All solution formulations, including the reference formulation, were well tolerated with most participants reporting injection-site pain of low severity (less than 10 mm). Across all time points (0 to 60 minutes post-injection) for all formulations, 76% to 100% of participants reported VAS pain scores of less than 10 mm and mean VAS pain scores ranged from 0.2 to 7.1 mm.

All solution formulations, including the reference formulation, administered by SC injection were well tolerated by participants. There were no deaths or SAEs. One participant was discontinued due to an AE that was not related to study intervention, as judged by the investigator. The frequency of AEs was low overall. All treatment-emergent adverse events (TEAE) were mild or moderate in severity and no TEAEs were related to study intervention, as judged by the investigator. Fewer participants reported ISRs at prespecified time points from 10 to 60 minutes and fewer spontaneously reported ISRs following injection of test formulations compared to the reference formulation. Mild pain was the most common ISR parameter reported at prespecified time points and for spontaneously reported events.

### Sequences

```
SEQ ID NO: 1
                10 20 30 40
FVNQHLCGSHLVEALELVCGERGFHYGGGGGGSGGGGGIV
                50 60 70 80
EQCCTSTCSLDQLENYCGGGGGGQGGGGQGGGGQGGGGGE
                90 100 110 120
CPPCPAPPVAGPSVFLFPPKPKDTLMISRTPEVTCVVVDV
              130 140 150 160
SHEDPEVQFNWYVDGVEVHNAKTKPREEQFNSTFRVVSVL
              170 180 190 200
TVVHQDWLNGKEYKCKVSNKGLPAPIEKTISKTKGQPREP
              210 220 230 240
QVYTLPPSREEMTKNQVSLTCLVKGFYPSDIAVEWESNGQ
              250 260 270 280
PENNYKTTPPMLDSDGSFFLYSKLTVDKSRWQQGNVFSCS
              290
VMHEALHNHYTQKSLSLSPG
```

1. An aqueous, sterile pharmaceutical composition comprising:
a) an insulin-Fc fusion;
b) phenol;
c) one or more additional preservatives selected from the group consisting of phenoxyethanol and benzyl alcohol;
d) a tonicity agent;
e) a surfactant; and
f) a buffer;
and having a pH between 6 to 7.5; and
wherein the phenol and one or more additional preservatives are present in concentrations that allow for an in-use period of at least 12 weeks without unacceptable loss of stability.

2. The composition of claim 1 wherein the concentration of phenol is from 1.5 to 4 mg/mL.

3. The composition of claim 1 wherein the concentration of phenol is about 1.8 mg/mL.

4. The composition of claim 1 wherein the additional preservative is phenoxyethanol.

5. The composition of claim 4 wherein the concentration of phenoxyethanol is between 4 and 14 mg/mL.

6. The composition of claim 4 wherein the concentration of phenoxyethanol is about 4 mg/mL.

7. The composition of claim 4 wherein the concentration of phenoxyethanol is about 8 mg/mL

8. The composition of claim 1 wherein the additional preservative is benzyl alcohol.

9. The composition of claim 8 wherein the concentration of benzyl alcohol is between 5 to 10 mg/mL.

10. The composition of claim 9 wherein the concentration of benzyl alcohol is about 9 mg/mL.

11. The composition of claim 1 wherein the insulin-Fc fusion is insulin efistora alfa.

12. The composition of claim 11 wherein the concentration of insulin efistora alfa is between 5 to 30 mg/mL.

13. The composition of claim 12 wherein the concentration of insulin efistora alfa BIF is selected from the group consisting of about 7.15, 14.3, and 28.6 mg/mL.

14. The composition of claim 1 wherein the pH of the composition is between 6.3 to 6.8.

15. The composition of claim 1 wherein the pH of the composition is about 6.5.

16. The composition of claim 1 wherein the tonicity agent is selected from the group consisting of sodium chloride, mannitol, and glycerin.

17. The composition of claim 1 wherein the tonicity agent is glycerin.

18. The composition of claim 17 wherein the concentration of glycerin is between 15 to 35 mg/mL.

19. The composition of claim 18 wherein the concentration of glycerin is about 17 mg/mL.

20. The composition of claim 1 wherein the surfactant is selected from the group consisting of poloxamer 188, polysorbate 20, and polysorbate 80.

21. The composition of claim 19 wherein the surfactant is poloxamer 188.

22. The composition of claim 21 wherein the concentration of poloxamer 188 is between 0.01 to 0.5 mg/mL.

23. The composition of claim 22 wherein the concentration of poloxamer 188 is about 0.4 mg/mL.

24. The composition of claim 1 wherein the buffer is selected from the group consisting of phosphate, citrate, and TRIS.

25. The composition of claim 24 wherein the buffer is phosphate.

26. The composition of claim 25 wherein the concentration of phosphate is between 5 to 10 mM.

27. An aqueous, sterile pharmaceutical composition comprising:
a) insulin efistora alfa in a concentration of 5 to 30 mg/mL;
b) phenol in a concentration of 1.5 to 4 mg/ml;
c) benzyl alcohol in a concentration of 4 to 14 mg/mL;
d) glycerin in a concentration of 15 to 35 mg/mL;
e) poloxamer 188 in a concentration of 0.01 to 0.5 mg/mL; and
f) phosphate in a concentration of 5 to 10 mM;
wherein the composition has a pH of 6.3 to 6.8.

28. The composition of claim 27 wherein:
a) insulin efistora alfa is in a concentration selected from the group consisting of about 7.15, about 14.3, and about 28.6 mg/mL;
b) phenol is in a concentration of about 1.8 mg/ml;
c) benzyl alcohol is in a concentration of about 9 mg/mL;
d) glycerin is in a concentration of about 17 mg/ml;
e) poloxamer 188 is in a concentration of about 0.4 mg/mL; and
f) phosphate is in a concentration selected from the group consisting of about 5 and about 10 mM;
wherein the composition has a pH of about 6.5.

29. The composition of claim 28, wherein the concentration of insulin efistora alfa is about 14.3 mg/mL and the concentration of phosphate is about 5 mM.

30. The composition of claim 28, wherein the concentration of insulin efistora alfa is about 28.6 mg/mL and the concentration of phosphate is about 10 mM.

31. The composition of claim 28, wherein the concentration of insulin efistora alfa is about 7.15 mg/mL.

32. A method of treating diabetes comprising administering to a human in need thereof an effective dose of the pharmaceutical composition of claim 1.

33. An article of manufacture comprising of the pharmaceutical compositions of claim 1.

34. The article of manufacture of claim 33 which is a multi-use vial.

35. The article of manufacture of claim 33 which is a multi-use pen injector.

36. The article of manufacture of claim 33 which is a pump device for continuous subcutaneous insulin infusion therapy.
